(19) United States
(12) Patent Application Publication
(10) Pub. No.: US 2010/0112565 A1
(43) Pub. Date: May 6, 2010

(54) Methods, kits, and reaction mixtures for high resolution melt genotyping

(75) Inventor: Andreas R. Tobler, Fremont, CA (US)

Correspondence Address: LIFE TECHNOLOGIES CORPORATION;C/O INTELLEVATE, P.O. BOX 52050, MINNEAPOLIS, MN, 55402, US

(73) Assignee: Life Technologies Corporation, Carlsbad, CA (US)

(21) Appl. No.: 12/384,027

(22) Filed: Mar. 31, 2009

Related U.S. Application Data
(63) Continuation-in-part of application No. 12/264,193, filed on Nov. 3, 2008.

(51) Int. Cl. C12Q 1/68

(52) U.S. Cl. 435/6

(57) ABSTRACT

Various methods are described that provide for high resolution melt (HRM) genotyping. Some embodiments comprise providing a locus specific primer, and two allele specific primers each comprising at least one single nucleotide polymorphism (SNP) allele-hybridizable sequence, wherein at least one of the allele specific primers also comprises at least one nucleotide alteration. In some embodiments, a nucleic acid is provided comprising a SNP base located within 1-20 bases of its 3′ end. Some embodiments comprise hybridizing the locus specific primer and at least one of the allele specific primers to the nucleic acid, amplifying the hybridized nucleic acid using pyrophosphorolysis activated polymerization (PAP) PCR, and determining the melting temperature (Tm) of the resulting amplicons, for example, using HRM. In some embodiments, reaction mixtures and kits for HRM genotyping are provided. The reaction mixtures and kits can each comprise a locus specific primer, one or more allele specific primers each comprising at least one SNP allele-hybridizable sequence, and a PAP PCR enzyme, wherein at least one of the allele specific primers also comprises a nucleotide alteration, for example, a tail.

### CROSS-REFERENCE TO RELATED APPLICATION

This application is a continuation-in-part of U.S. patent application Ser. No. 12/264,193, filed Nov. 3, 2008, which is incorporated herein in its entirety by reference.

### BACKGROUND

High resolution melting (HRM) is a homogeneous, close-tube, post-PCR method that goes beyond the classical melting curve analysis by enabling scientists to study genetic variation and thermal denaturation of nucleic acids in much more detail and with much higher information yield. The problem with most HRM methods is that they are not universally applicable to a variety of measurements or genotyping. What is needed is a more universal method, kit, and reaction mixtures that can enable HRM analysis of more genetic variations, including single nucleotide polymorphisms (SNPs) and SNPs comprising base pair inversions, with higher efficiency and accuracy, independent of the specific nature of the genetic variation in a sample.

### SUMMARY

Various embodiments provide methods for high resolution melt (HRM) genotyping. In some embodiments, the method can comprise hybridizing a locus specific primer and at least one allele specific primer to a nucleic acid-containing sample comprising a SNP base located within 1 to 20 bases of the 3′ end of the nucleic acid, and amplifying the hybridized nucleic acid using polymerase chain reaction (PCR). In some embodiments, the PCR can comprise an activatable PCR, for example, pyrophosphorolysis activated polymerization (PAP) PCR. In some embodiments, an activatable nucleotide can be used to control the reaction. The methods can comprise providing a locus specific primer, providing two allele specific primers each comprising at least one SNP allele-hybridizable sequence and at least one of them comprising a nucleotide alteration, providing a nucleic acid comprising a SNP base located within 1-20 bases of the 3′ end of the nucleic acid, hybridizing the locus specific primer and at least one allele specific primer to the nucleic acid, amplifying the hybridized nucleic acid using PAP PCR to produce amplicons, and determining the melting temperature (Tm) of the amplicons using a melting curve analysis, for example, using HRM. The SNP allele-hybridizable sequence can, for example, be designed to probe for an allele, for example, to probe for a SNP allele. The at least one nucleotide alteration can comprise one or more nucleotides that differ from nucleotides that would otherwise render the allele specific primer fully complementary to the nucleic acid. In some embodiments, the nucleotide alteration can comprise a tail on the primer, for example, a tail comprising two to five nucleotides.

In some embodiments, reaction mixtures and kits for HRM genotyping are provided. The reaction mixture for HRM genotyping can comprise a locus specific primer and one or more allele specific primers. Each allele specific primer can comprise at least one SNP allele-hybridizable sequence and at least one of the allele specific primers can comprise a nucleotide alteration. The reaction mixtures can optionally comprise one or more nucleic acids, and/or one or more PAP PCR enzymes, and/or one or more controls or standards.

In some embodiments, the kit can comprise a locus specific primer, one or more allele specific primers, and one or more PAP PCR enzymes. Each of the allele specific primers can comprise at least one SNP allele-hybridizable sequence, and at least one of the allele specific primers can comprise a nucleotide alteration, for example, a tail. The kit can comprise two or more allele specific primers designed to probe for each allele of a SNP allele. In some embodiments, each allele specific primer comprises a nucleotide alteration. In some embodiments, the kit can comprise a nucleic acid and the nucleic acid can comprise a sample, for example, a heterozygous sample comprising at least two different alleles of a SNP-containing nucleic acid. In some embodiments, the nucleic acid can comprise a sample that is homozygous for one allele. The kit can comprise one or more controls or standards.

These and other features of the present teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows a block diagram of a general thermocycler and HRM instrument according to various embodiments of the present teachings.

FIG. 2 shows a flow chart of general methods employed according to various embodiments of the present teachings.

FIG. 3 shows a diagram of a locus specific primer and two allele specific primers, how they are used with PAP PCR to amplify nucleic acids for HRM analysis, and the two different respective melting curves of the amplicons according to various embodiments of the present teachings.

FIG. 4A is a table showing three different examples of possible allele combinations that can result from a nucleic acid that comprises a SNP base, and which can be detected using HRM techniques according to various embodiments of the present teachings.

FIG. 4B shows the associated HRM melt curves for each of Examples 1-3 shown in FIG. 4A.

FIG. 5A is a table showing three different examples of possible allele combinations resulting from a nucleic acid that comprises a base pair inversion wherein the alleles have been probed for using allele specific primers that do not comprise nucleotide alterations.

FIG. 5B shows the associated HRM melt curves for each of Examples 4-6 shown in FIG. 5A.

FIG. 6A is a table showing examples of the same three possible allele combinations shown in FIG. 5A, but wherein the alleles have been probed for using an allele specific primer that comprises a nucleotide alteration according to various embodiments of the present teachings.

FIG. 6B shows the associated HRM melt curves for each of Examples 7-9 shown in FIG. 6A.

### DESCRIPTION OF VARIOUS EMBODIMENTS

For the purpose of interpreting this specification, the following definitions will apply and whenever appropriate, terms used in the singular will also include the plural and vice versa. It is noted that, as used in this specification and the appended claims, the singular forms “a,” “an,” and “the,” include plural referents unless expressly and unequivocally limited to one referent. Thus, for example, reference to a “primer” includes more than one “primer”. Reference to “genomic DNA” can refer to more than one strand of “genomic DNA”. The use of “or” means “and/or” unless stated otherwise. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.” In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The section headings used herein are for organizational purposes only and are not to be construed as limiting the subject matter described in any way.

In describing and claiming the embodiments, the following terminology will be used with the definitions set out below.

The abbreviations for the various nucleic acid bases include guanine (G), thymine (T), adenine (A), and Cytosine (C).

The term “allele specific primer” refers to a primer that binds to a specific sequence on a region of a nucleic acid to be amplified. These types of primers can be used to amplify and discriminate between two or more alleles of a gene, for example, simultaneously. The difference between the two alleles can be a SNP, an insertion, or a deletion. One or each allele specific primer can independently comprise a base sequence comprising a tail. In some embodiments, at least one of the allelele specific primers does not comprise a tail. For instance, at least one SNP allele-hybridizable sequence and at least one nucleotide alteration can be provided in a base sequence of one or more of the allele specific primers. In various embodiments the 3′ end of the allele specific primer can be blocked by techniques known in the art, such that the blocked end can be activatable by a PAP PCR enzyme or similar type of enzyme.

The term “amplicons” refers to portions of nucleic acid that have been amplified or multiplied using a technique such as the polymerase chain reaction (PCR) methodology.

The term “base sequence” refers to the nucleotide sequence of one or more allele specific primers without the addition of a tail or a nucleotide alteration. The “base sequence” in many cases is a known, established or predicted sequence of the allele specific primer. This term is distinguished from terms in the art that may apply the term “base sequence” to further comprise a tail or a nucleotide alteration.

The term “computer” refers to all the associated hardware, software, processors, and displays to perform data acquisition and analysis.

The term “genomic DNA” refers to the total DNA from an organism. That is, the whole complement of an organism's DNA. Typically, this includes both the intron and exon sequences and the non-coding regulatory sequences such as the promoter and enhancer sequences.

The term “high resolution melt” or “HRM” refers to a technique for determining a sequence variation in a nucleic acid by analyzing a melting curve of the nucleic acid. The nucleic acid can be double-stranded or single-stranded. In some embodiments, a signal representing a double-stranded nucleic acid can be measured in real time. In some embodiments, real time measurement (Ct) of the PCR can be used as a quality control value for melt analysis such as HRM analysis. In some embodiments, end point analysis can be employed. In some embodiments, single-stranded nucleic acids can be analyzed, for instance, single-stranded nucleic acids can fold to form duplexes or other higher ordered structures that can then be analyzed. Herein, the term “high resolution melt” or “HRM” can also refer to a technique for determining sequence variations between two different nucleic acids by analyzing the shape of the melting curve including the melting temperature and the slope.

The term “locus specific primer” refers to a primer that binds to a particular region of a nucleic acid to be amplified. Generally an allele specific primer and a locus specific primer are utilized to perform PCR on leading and lagging strands of a DNA or template strand and complement. In various embodiments, the 3′ end of the locus specific primer can be blocked by techniques known in the art. The blocked end can be activated by a PAP PCR enzyme or similar type of enzyme. In various embodiments, a locus specific primer can already be hybridized to a nucleic acid before the hybridized nucleic acid is amplified, such that a method is provided that does not require a step of hybridizing a locus specific primer.

The term “nucleic acid” or “nucleic acid strand” refers to a single-stranded or double-stranded DNA or cDNA, or versions of the same, produced or processed from any type of nucleic acid. For instance, DNA, cDNA, RNA, mRNA, tRNA, or modified or derivitized versions of the same. Herein, “nucleic acid” can also refer to a mixed sample comprising two or more alleles of a nucleic acid. Further, the nucleic acid can comprise a 3′ end comprising a single nucleotide polymorphism base located, for example, within 1 to 20, or 1 to 15, or 1 to 10, or 5 to 10, or 10 to 20, or 10 to 15 nucleotides of the 3′ end. In various embodiments, the 3′ end can be blocked and capable of activation by a PAP PCR enzyme. Blocking techniques and activatable oligonucleotides that can be used are well known in the art.

The term “nucleotide alteration” refers to a change in one or more nucleotides in one or more base sequences of an allele specific primer, or by way of addition of one or more nucleotides to the ends of one or more allele specific primers by way of a “tail.” A “nucleotide alteration” can be a change in a nucleotide base from a known or predicted primer sequence, from an unknown primer sequence, or from a partially known primer sequence. For example, base sequence alterations to one or more allele specific primers can comprise the addition of from 1 to 5 nucleotides, for example, from 2 to 4 nucleotides. This is by way of example and should not be interpreted to limit the scope of possible nucleotide changes. For instance, it is within the scope of the present teachings that additional nucleotide changes can be used to the extent that the allele specific primer or primers maintain functionality. The one or more nucleotide alterations can be made to an allele specific primer or primers by way of a tail, by way of making the allele specific primer longer, or by way of a combination thereof. The nucleotide alteration can comprise the addition of from 1 to 30 nucleotides, for example, the addition of 1 to 20 nucleotides, 1 to 10 nucleotides, or 2 to 5 nucleotides, added to the 5′ end of the allele specific primer. One or more of the added nucleotides can be complementary to the target polynucleotide. In some embodiments, the entirety of the added nucleotides can be non-complementary to the target polynucleotide. Further, the teachings should not be interpreted to be limited to these examples. It is within the scope of the teachings that nucleotide alterations can be used to the extent that the allele specific primers maintain their functionality.

The term “primer” refers to an oligonucleotide or single-stranded nucleic acid that hybridizes with a complementary portion of another single-stranded molecule as a starting point for initiation of polymerization mediated by an enzyme with DNA polymerase activity. According to various embodiments, typing methods can be based on amplification of specific genes from genomic DNA using polymerase chain reaction (PCR). The PCR amplification of genes can involve the use of locus specific, group specific, and/or allele specific primers. Locus specific primers can be used to amplify all alleles encoded at a given locus but not alleles encoded by other loci. Allele specific primers amplify families of alleles that share a common polymorphism. Allele specific primers can be used to amplify a single allele and can differentiate between two sequences that differ by only a single base difference or change. According to various embodiments, amplification methods can comprise using combinations of locus specific primers to amplify and analyze both alleles in a heterozygous sample, followed by allele specific amplification to isolate one of the two alleles for further characterization. In some embodiments, two locus specific primers can be used in a preamplification reaction before a genotyping reaction is carried out.

The term “PAP PCR enzyme” refers to any enzyme that can perform PAP polymerization reactions (called pyrophosphorolysis activated polymerization chain reaction). The PAP method can be used to amplify either RNA or DNA. When used to amplify DNA, the activatable oligonucleotide can comprise a 2′-deoxyoligonucleotide, the non-extendible 3′ terminus can comprise, for example, a 2′,3′-dideoxynucleotide or an acyclonucleotide or other blockers as known in the art, the four nucleoside triphosphates can comprise 2′-deoxynucleoside triphosphates or their analogs, and the nucleic acid polymerase can comprise a DNA polymerase. In some embodiments, the DNA polymerase used can also have pyrophosphorolysis activity. Some DNA polymerases having pyrophosphorolysis activity are thermostable Tfl, Taq, and genetically engineered DNA polymerases, such as AMPLITAQFS™, available from Applied Biosystems, Foster City, Calif., and THERMOSEQUENASE™, available from GE Healthcare Bio-Sciences Corp., Piscataway, N.J. These genetically engineered DNA polymerases have the mutation F667Y or an equivalent mutation in their active sites. The use of genetically engineered DNA polymerases, such as AMPLITAQFS™ and THERMOSEQUENASE™, can greatly improve the efficiency of PAP. These Family I DNA polymerases can be used according to various embodiments when the activatable oligonucleotide is a 3′ dideoxynucleotide or an acyclonucleotide. When the activatable oligonucleotide is an acyclonucleotide, Family II archaeon DNA polymerases can be used. Examples of such polymerases include, but are not limited to, Vent (exo-) and Pfu (exo-). These polymerases efficiently amplify 3′ acyclonucleotide blocked activatable oligonucleotides. In some embodiments, two or more polymerases can also be used in one reaction. If the template is RNA, the nucleic acid polymerase can comprise RNA polymerase, reverse transcriptase, their variants, or a combination thereof. The activatable oligonucleotide can comprise a ribonucleotide or a 2′-deoxynucleotide. The non-extendible 3′ terminus can comprise a 3′ deoxyribonucleotide or an acyclonucleotide. The four nucleoside triphosphates can comprise ribonucleoside triphosphates, 2′ deoxynucleoside triphosphates, or their analogs. For convenience, the description that follows uses DNA as a template. Processing RNA, however, is also well within the scope of the present teachings. Further, details, uses, and methods regarding these enzymes can be found in U.S. Pat. No. 7,033,763, which is incorporated herein by reference in its entirety.

The term “pyrophosphorolysis activated polymerization” (PAP) refers to a reaction that works in a reverse reaction to DNA polymerization and results in the removal of the 3′ terminal nucleotide of an annealed oligonucleotide.

The term “single nucleotide polymorphism” (SNP) refers to a DNA sequence variation occurring when a single nucleotide, e.g., A, T, C, or G, in the genome (or other shared sequence) differs between members of a species (or between paired chromosomes in an individual). For example, two sequenced DNA fragments from different individuals, AAGCCTA to AAGCTTA, contain a difference in a single nucleotide. In this case it is to be understood that there are two alleles: C and T. Almost all common SNPs have only two alleles.

The term “tail” refers to any desired number and type of nucleotide bases or additions of any kind that change the Tm of the amplicon. For instance, one or more nucleotides can be added to the ends of one or more allele specific primers or to the ends of base sequences of allele specific primers. The number of added nucleotides can, for example, be in a range of from 1 to 30 nucleotides added to one or more of the allele specific primers or added to one or more base sequences of the allele specific primers. In various embodiments, the number of added nucleotides can be, for example, in a range of from 1 to 25, from 1 to 20, from 1 to 15, from 1 to 10, from 5 to 20, from 10 to 20, or from 10 to 15 nucleotides. All, none, or a portion of a tail added to an allele specific primer can be complementary to a target polynucleotide.

In some embodiments, a melting curve analysis is performed on double-stranded DNA samples resulting from real-time PCR in a target DNA region in which a mutation of interest lies. The analysis can comprise an HRM analysis. A warming of the amplicon DNA from around 50° C. to around 95° C. causes the two strands of DNA to melt and the dissociation can be characteristically measured. In practice, the melting process is monitored at high resolution and in real time. In some embodiments, a fluorescent dye can be used as an indicator of the dissociation. In various embodiments, in a measurement of the melt of a double-stranded DNA, an intercalating dye can be used that binds specifically to double-stranded DNA and fluoresces at a characteristic and measurable level as long as binding to the double-stranded DNA is maintained. At the melting temperature for a particular double-stranded entity, the fluorescence changes. For example, a high level of fluorescence indicates a population of double-stranded entities in the sample, as the temperature rises the two strands of the double-stranded DNA separate and the fluorescence changes accordingly. High resolution detection of the melting event yields a melting curve and a melting temperature (Tm) that show the change in fluorescence as a function of temperature. According to various embodiments, the detection can be used to reveal differences in sequence variants, for example, to determine if a sample is homozygous for a first allele of a gene, homozygous for a second allele of the gene, or heterozygous.

Various embodiments of the present teachings are described with reference to the accompanying figures. In some instances, the figures are not to scale and have been exaggerated for clarity of presentation. According to various embodiments, allele specific PAP PCR with tailed primers or primers having other nucleotide alterations, followed by melting curve analysis, provides an assay that can be applied to the analysis of most, and in many instances all, SNPs, not just to a subset of SNPs. The resolution of a SNP assay can be greatly increased by adjusting alleles specifically to the length and sequence of a desired PCR amplicon. According to various embodiments, an HRM assay can be used in a quantitative way, e.g., for allele-quantification of SNPs, because the melt curves of the two allele specific amplicons are clearly separated. According to various embodiments, the high specificity of PAP PCR is used to greatly reduce the risk of non-specific PCR amplification and to increase specificity as well as sensitivity of HRM assays.

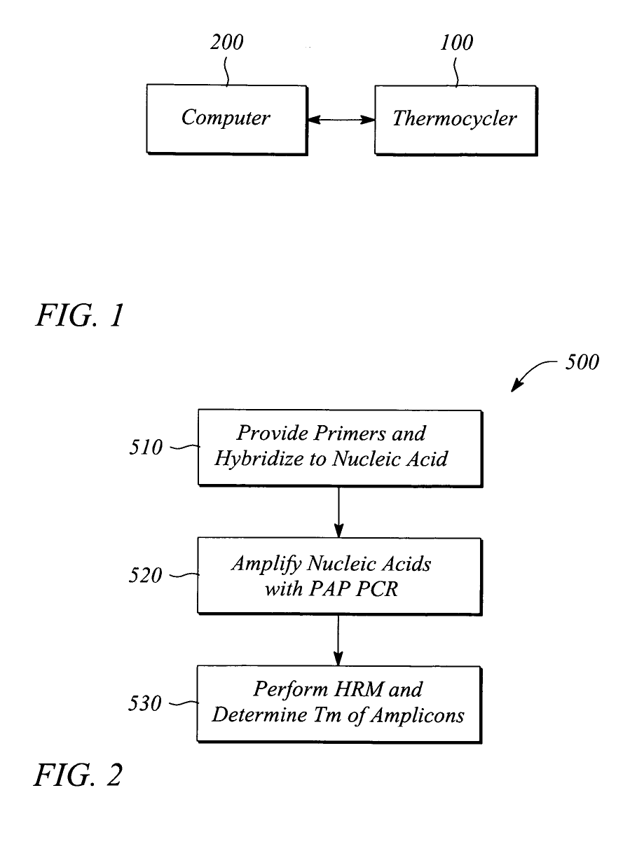

According to various embodiments, HRM-based sequence analysis is used as a powerful technology for SNP genotyping and mutation scanning. Allele specific PAP PCR with tailed primers or primers having other nucleotide alterations, followed by HRM analysis according to various embodiments of the present teachings, converts an HRM platform into a robust and quantitative mutation screening platform capable of analyzing any SNP. An increased allele specific resolution between PCR amplicons allows quantitative genotyping applications like allele quantitation, or allele specific gene expression analysis. The present robust assay platform can be used in assays for clinical research as well as for diagnostic applications. Having generally discussed various embodiments, a more detailed description is now in order. Referring now to FIG. 1, various embodiments will now be described in more detail.

FIG. 1 shows a real time thermocycler instrument 100 with high resolution melt (HRM) capability that can be used according to various embodiments of the present teachings. Various types of thermocyclers have been described in the literature to perform PCR. Some types of thermocyclers with HRM that can be employed according to various embodiments include, but are not limited to: the AB 7300, available from Applied Biosystems, LLC., Foster City, Calif.; the HR-1™, available from Idaho Technologies, Inc., Salt Lake City, Utah; the LIGHTCYCLER 480®, available from Roche Diagnostics Corporation, Indianapolis, Ind.; the MASTER CYCLER®, available from Eppendorf, Hamburg, Germany; the LIGHTSCANNER®, available from Idaho Technologies, Inc., Salt Lake City, Utah; and the ROTOR-GENE™ available from Corbett Life Science, Sydney, Australia. Each of these instruments can be used to enable a real time PCR reaction followed by HRM. Thermocycler 100 can be employed with various types of computers 200 for various HRM analyses. Generally, thermocycler 100 performs a number of PCR amplification reactions. After these reactions have been completed, the results are subjected to HRM to generate one or more melt curves. Each HRM melt curve can be displayed on computer 200 or on another similar type of device having a user interface. The data and results can be calibrated and displayed using software. Software can be present in computer 200 or on a computer readable medium.

According to various embodiments, different genetic sequences are caused to melt at slightly different temperatures and/or rates, and representative signals can be viewed and compared using melting curves, such that the different genetic sequences can be detected. Even a single base difference can cause differences in a melting curve. The process can be used, for example, for specific genotyping, comparing sequence identities between two DNA samples, scanning for sequence variants between two primers, or a combination thereof. According to various embodiments, high-resolution DNA melting is used to quickly and accurately determine whether DNA sequences match, providing an interesting option for transplantation matching and forensics. Genotyping via high-resolution melting, according to various embodiments, can be more streamlined and less expensive than methods that use complex probes, as described, for example, in: Ririe et al., 1997, Product differentiation by analysis of DNA melting curves during the polymerase chain reaction, Anal Biochem. 245: 154-160; and Wittwer et al, 2003, High-Resolution genotyping by amplicon melting analysis using LCGreen, Clin. Chem. 49: 853-860; each of which is incorporated herein by reference in its entirety.

FIG. 2, illustrates a flow chart of methods according to various embodiments. A general method for HRM 500 can comprise providing various primers and hybridizing to a nucleic acid, as depicted at step 510, to form a hybridized nucleic acid. The method can then involve amplifying the hybridized nucleic acid with a PAP PCR enzyme as depicted at step 520. The method can then involve performing HRM to determine the Tm of amplicons as depicted at step 530.

Pyrophosphorolysis activated polymerase PCR (PAP PCR) comprises the reverse reaction of DNA polymerization and results in the removal of the 3′ terminal nucleotide of an annealed oligonucleotide. Primers used for PAP PCR can be blocked at their 3′ ends and have to be activated by pyrophosphorolysis for extension to occur. The activation of a 3′ blocked primer can comprise a very specific event, sensitive to mismatches not only at the 3′ end, but also with the primer. For example, a mismatch within at least 1 to 20 base pairs of the 3′ end, can essentially block activation. This property can be exploited to increase the Tm difference between two different amplicons in an allele specific way.

The power of HRM to determine genetic variations and provide high resolution can be facilitated by the addition of a dye to the reaction mixture or sample. Various generic or intercalating dyes are known in the art which can be used for HRM analysis. Some of these dyes comprise cyanine based dyes. Some dyes will bind to single-stranded DNA, or to double-stranded DNA, or will intercalate into base pairs of DNA. Examples of dyes in present use include, but are not limited to: SYTO9®, a green fluorescent DNA intercalating dye, available from Molecular Probes, Eugene, Oreg.; EVA GREEN™, a green fluorescent double-stranded DNA (dsDNA) binding dye, available from Biotium, Hayward, Calif.; BEBO, an unsymmetric cyanine based, dsDNA binding dye, available from TATAA Biocenter, Goteborg, Sweden; SYBR®GREEN, a green fluorescent dsDNA binding dye, available from Molecular Probes, Eugene, Oreg.; and LCGREEN® a green fluorescent dsDNA binding dye, available from Idaho Technologies, Inc, Salt Lake City, Utah. It is within the scope of the present teachings that other types of molecules, dyes, compounds, or mixtures can be added to the reaction mixture or sample to help in determining various genetic variations in nucleic acids. Some of these reaction mixtures, dyes, compounds, or molecules are known in the art.

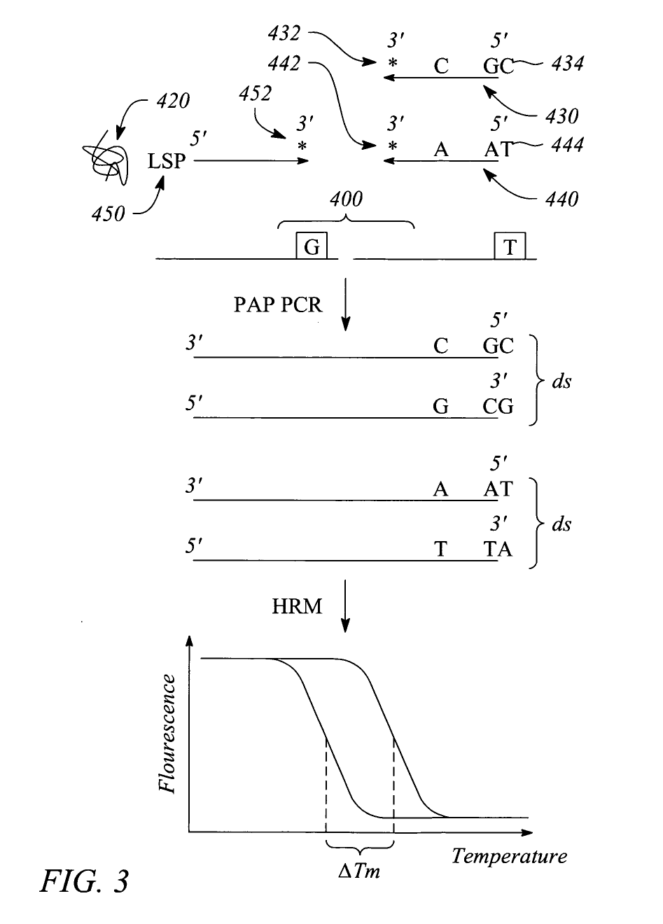

FIG. 3 shows both alleles of a nucleic acid 400 that can be used for PAP PCR. Nucleic acid 400 can comprise cDNA or DNA or versions of the same derived or processed from any type of nucleic acid. In certain instances, the nucleic acid can comprise genomic DNA (gDNA as shown in the figure) from a single organism. In some cases, the genomic DNA can comprise a mixture of nucleic acids or nucleic acids from various organisms. Nucleic acid 400 can comprise, for example, up to 1000 bases in single-stranded form, and in some embodiments up to 200 bases or up to 60 bases. In various embodiments, genotyping can be accomplished for both known and unknown portions of the nucleic acid. In many instances, however, the SNP of interest can be located in or around a region of the nucleic acid to which the primers are targeted. In addition, the position of the SNP may or may not be known. In the present example, both alleles are shown in single-stranded form and a known G/T SNP is shown such that the allele on the left comprises a G at the boxed-in SNP location and the allele on the right comprises a T at the boxed-in SNP location. Both alleles of nucleic acid 400 exhibit the G/T SNP base near the 3′ end of the DNA.

As provided in various embodiments, various types of SNPs can be provided or present in the nucleic acid and/or probed for according to various embodiments. It is within the scope of the various embodiments that more than one SNP base can be present and more than one SNP allele specific primer can be used. The SNP base can be located near the 3′ end of a single stranded nucleic acid, to facilitate allele specific amplification, for example, located within 1 to 20 bases, within 1 to 15 bases, within 1 to 10 bases, within 1 to 5 bases, or within 5 to 10 bases of the 3′ end. The exact location is not a requirement, but can be used to select a specific enzyme that can be used. For instance, the PAP PCR enzyme has been shown to be allele specific and does not typically allow for PCR extension when there are extensive mismatches in base pairing. Further, a mismatch or nucleotide alteration can be provided within the strand length of the allele specific primer, and in various embodiments, this can comprise the first 1-20 bases of a single-stranded allele specific primer, the first 1-20 bases of a complementary nucleic acid, or the first 1-20 base pairs of a double-stranded allele specific primer. Various embodiments exploit this enzyme specificity. See, for example, Liu and Sommer, 2004, PAP: Detection of ultra rare mutations depends on P* oligonucleotides: sleeping beauties awakened by the kiss of pyrophosphorolysis, Human Mutation 23:426-436; which is incorporated herein in its entirety by reference.

Various PAP PCR enzymes are known and used in the art, as described, for example, in Liu and Sommer, 2004, PAP: Detection of ultra rare mutations depends on P* oligonucleotides: sleeping beauties awakened by the kiss of pyrophosphorolysis, Human Mutation 23:426-436; Liu and Sommer, 2004, Pyrophosphorolysis by Type II DNA polymerases: implications for pyrophosphorolysis-activated polymerization, Anal Biochem 324(1):22-28; Liu and Sommer, 2000, Pyrophosphorolysis-activated polymerization (PAP): application to allele-specific amplification, Biotechniques 29(5):1072-1080, each of which is incorporated herein in its entirety by reference.

It should be noted that although a PAP PCR enzyme 420 can be employed according to various embodiments, other enzymes or variations of the PAP PCR enzyme can be used. According to various embodiments, enzymes can be chosen based on their capability of extending blocked primer ends only upon proper base pair matching in the nucleic acids and the SNP. Other enzymes that are similar or different to the PAP PCR enzyme are within the scope of the various embodiments. In some embodiments, the use of the PAP PCR enzyme relies on a variant of the enzyme that maintains the substantial enzymatic function of the original.

The present teachings also contemplate functional portions of the enzyme. As used herein, the “functional portion” of an enzyme is that portion which contains the active site essential for effecting the catalytic step, i.e. the portion of the molecule that is capable of binding one or more reactants or is capable of improving or regulating the rate of the PCR reaction. The active site can be made up of separate portions present on one or more polypeptide chains and can generally exhibit high substrate specificity. The teachings also include functional equivalents of the enzyme described above. The enzyme can comprise an equivalent protein that can be considered a functional equivalent of an original protein for a specific function if the equivalent protein is immunologically cross-reactive with, and has the same function as, the original protein. The equivalent protein can, for example, comprise a fragment of an original protein, or a substitution, addition, or deletion mutant of an original protein. For example, it is possible to substitute amino acids in a sequence with equivalent amino acids using conventional techniques. Groups of amino acids known normally to be equivalent are:
(a) Ala(A), Ser(S), Thr(T), Pro(P), Gly(G);
(b) Asn(N), Asp(D), Glu(E), Gln(Q);
(c) His(H), Arg(R), Lys(K);
(d) Met(M), Leu(L), Ile(I), Val(V); and
(e) Phe(F), Tyr(Y), Trp(W).

Substitutions, additions and/or deletions in the enzyme can be made as long as the resulting equivalent enzyme is immunologically cross-reactive with, and has essentially the same function as, the native enzyme. The equivalent enzymes will normally have substantially the same amino acid sequence as the native enzyme. An amino acid sequence that is substantially the same as another sequence, but that differs from the other sequence by means of one or more substitutions, additions, and/or deletions is/are considered to be an equivalent amino acid sequence. For example, less than 25%, or less than 10%, or less than 5% of the number of amino acid residues in the amino acid sequence of the native enzyme can be substituted for, added to, or deleted from the native enzyme while still remaining an equivalent enzyme.

FIG. 3 shows a first allele specific primer 430 and a second allele specific primer 440 that are employed according to various embodiments. First allele specific primer 430 and second allele specific primer 440 can comprise any suitable number of nucleotides and lengths that enable hybridization to a target nucleic acid with specificity. It is within the scope of various embodiments that various primer types can be employed.

First allele specific primer 430 can comprise a blocked 3′ end 432. Blocked end 432 can be blocked in any number of different ways known in the art. Blocking can be accomplished using chemical modification, base pair alteration, and the like. Blocked end 432 can be designed to prevent normal PCR extension of the primer during amplification. For instance, blocked end 432 can comprise a dideoxy end that is blocked from providing normal PCR extension and amplification. Blocked end 432 can be removable under desired conditions.

First allele specific primer 430 can comprise a 5′ end that comprises a first tail 434. First tail 434 can comprise any desired number and types of nucleotide bases, or additions of any kind, that change the Tm of an amplicon resulting from hybridization with, and subsequent amplification of, a target nucleic acid. In various embodiments, the number of nucleotides that can be present in first tail 434 can be in a range of from 1 to 25, from 1 to 20, from 1 to 15, from 1 to 10, from 2 to 15, or from 10 to 15 nucleotides.

In FIG. 3, first tail 434 is shown as a GC sequence. The sequence in various embodiments can comprise more nucleotides, and in some instances, at least 10 more nucleotides. As will be discussed below, first tail 434 can be important in helping to distinguish a type of SNP present in a nucleic acid. This is mainly accomplished by the different Tm's that are determined using HRM.

First allele specific primer 430 can comprise a known nucleotide position shown as a C, which is used to probe for a particular SNP allele in the nucleic acid. For instance, in this case, first allele specific primer 430 can probe for the SNP allele that contains a G at the SNP location boxed-in in the single-stranded nucleic acid labeled gDNA.

Second allele specific primer 440 can comprise a blocked 3′ end 442. Blocked end 442 (all blocked ends are shown in the FIGS. with an asterisk (*)) can be blocked in any number of different ways known in the art. As explained above, this can be accomplished using chemical modification, base pair alteration, and the like. Blocked end 442 can be designed to prevent normal PCR extension of the primer during amplification. For instance, blocked end 442 can comprise a dideoxy end that can be blocked from providing normal PCR extension and amplification. Blocked end 442 can be removable under desired conditions.

Second allele specific primer 440 can comprise a 5′ end that comprises a second tail 444. Second tail 444 can comprise any desired number and type of nucleotide bases, such as previously described herein, or additions of any kind, that change the Tm of an amplicon resulting from hybridization with, and subsequent amplification of, a target nucleic acid. In various embodiments, the number of added nucleotides that can be present in second tail 444 can comprise, for example, from 1 to 25, from 1 to 20, from 1 to 15, from 1 to 10, from 2 to 15, or from 10 to 15 nucleotides. It is noted that in certain embodiments second tail 444 of second allele specific primer 440 can differ in length and/or nucleotide sequence from first tail 434 of first allele specific primer 430. In FIG. 3, second tail 444 is show as an AT sequence. The sequence in various embodiments can comprise more than two nucleotides, and in some instances, can comprise at least 10 nucleotides. As will be discussed below, second tail 444 can be used according to various embodiments to distinguish the SNP allele present in the nucleic acid by correlating the measured difference in Tm to the SNP allele present in the target nucleic acid.

In some embodiments, second allele specific primer 440 can comprise a known nucleotide position shown as A (in FIG. 3) that can be used to probe for a particular SNP alteration in the nucleic acid. For instance, in this case, second allele specific primer 440 can probe for the SNP allele that contains a T at the SNP location shown boxed-in in the single-stranded nucleic acid labeled gDNA.

Additional allele specific primers can be employed according to various embodiments and the teachings should not be interpreted to be limited to the specific examples provided. In addition, the sequences of the tails of the allele specific primers can be similar or different. In some embodiments, first allele specific primer 430 and second allele specific primer 440 can have the same sequence except for at the location complementary to the SNP location, and a tail is employed to distinguish the first allele specific primer from the second allele specific primer. In other embodiments, additional changes or alterations (over and above the differences at the location complementary to the SNP location) can be made to the base sequence of one or more of the allele specific primers to alter the Tm's so that each allele specific primer and their associated amplicons can be determined. For instance, if first allele specific primer 430 comprises a nine nucleotide base sequence of 5′TGACCGCCG3′ and second allele specific primer 440 comprises a nine nucleotide base sequence of 5′TGACCGCCT3′ (note that the SNP is underlined), one or more additional nucleotide changes internal to these base sequences can provide enough difference to the Tm's to distinguish first allele specific primer 430 from second allele specific primer 440. Further, as discussed above, one or more nucleotides can be added to one or more ends of one or more of the allele specific primers. These additions are called “tails”. The top of FIG. 3 shows the two-nucleotide tail GC added to the 5′ end of first allele specific primer 430, and the two-nucleotide tail AT added to the 5′ end of second allele specific primer 440.

FIG. 3 shows a locus specific primer 450 comprising a blocked end 452. Although FIG. 3 shows a blocked end (452), it is to be understood that in some embodiments the locus specific primer is not blocked at the 3′ end. As shown in FIG. 3, a primer such as locus specific primer 450 can be employed to allow for PCR extension of one or more hybridized nucleic acid strands. Locus specific primer 450 can comprise any suitable number of nucleotide bases that enables hybridization to a target nucleic acid, with specificity. It is within the scope of various embodiments to provide various types of locus specific primers of various lengths and sequences. Blocked end 452 can be blocked in any number of different ways known in the art. This can be accomplished using chemical modification, base pair alteration, and the like. Blocked end 452 is designed to prevent normal PCR extension of the primer during amplification. For instance, blocked end 452 can be a dideoxy end that is blocked from providing normal PCR extension and amplification. Blocked end 452 can be removable under desired conditions so that PCR extension can occur.

First allele specific primer 430, second allele specific primer 440, locus specific primer 450, and an optional nucleic acid 400 (DNA, cDNA, or versions of the same derived or processed from any type of nucleic acid) can be packaged individually or mixed together to make a kit (kit not shown in FIGS). An optional control or standard can be provided as part of the kit, for example, a known dye, a known probe, or one or more known alleles of a SNP-containing nucleic acid. The kit can be designed in any number of ways or combinations. The kit can comprise various types of packaging, e.g., a first container, and a second container, a box, a sealed package, and the like. It is also within the scope of the present teachings that instructions can be provided with the kit.

It is within the scope of various embodiments that certain reaction mixtures can be provided that comprise various combinations of locus specific primer 450, first allele specific primer 430, and second allele specific primer 440. An optional nucleic acid 400 can optionally be present in the reaction mixture. A control or standard can also be provided, for example, comprising a known dye, a known probe, or a known allele of a SNP-containing nucleic acid.

It should be noted that about 84% of all human SNPs result in A:T to G:C interchange with a Tm difference of approximately 1° C. in amplicons produced without using tailed primers. In 16% of SNPs, the SNP comprises a single base pair inversion (e.g., from A:T to T:A, or from G:C to C:G) and the Tm difference is smaller, for example, a Tm difference of only about 0.1° C. in amplicons produced using allelele specific primers without tails or other nucleotide alterations. A robust HRM assay, however, can exhibit a large Tm difference between genotypes, and it can be capable of analyzing more types of SNPs than could be analyzed by previous methods. This can be achieved, for example, by performing allele specific PAP PCR using tailed primers and/or primers having other nucleotide alterations, followed by HRM analysis, according to the present teachings.

With reference again to FIG. 3, the use of first tail 434 on first allele specific primer 430, and of second tail 444 on second allele specific primer 440, facilitates clearly distinguishing the melting of one allele from the melting of another. In some embodiments, first tail 434 and second tail 444 affect the overall Tm of each allele amplicon in such a way that the overall ΔTm between the two allele products is increased, or more easily distinguishable. This makes it easier to distinguish a unique situation exhibited for each allele of the SNP. For simplicity, FIG. 3 shows a comparison of the melting curve of a sample that is homozygous for the “G” SNP allele versus the melting curve of a sample that is homozygous for the “T” SNP allelele. As will be understood, both curves would not result from melting a single sample but rather FIG. 3 shows the two respective melting curves superimposed on one another. If a sample were instead heterozygous, a melting curve of different slope and shape would result as is described below. Other methods and embodiments and the analysis of heterozygous SNPs are also a part of the present teachings.

FIG. 3 shows nucleic acid 400 as both alleles of a G/T SNP located near the 3′ end of the single-stranded gDNA target nucleic acid. Nucleic acid 400 is not heterozygous but rather the two alleleles are shown as alternatives for the purpose of determining the ΔTm between them.

When a nucleic acid has an A/T SNP or a G/C SNP, it is said that the nucleic acid is heterozygous but differs by only a base pair inversion. When the nucleic acid has a G/A SNP, a G/T SNP, a C/A SNP, or a C/T SNP, the nucleic acid is also heterozygous but the result is a difference in hydrogen bonding in the two alleles (i.e. from three bonds to two, or vice versa). This change in hydrogen bonding impacts the overall Tm of resulting amplicons and results in two very different melting curves. The difference in the melting curves can be enhanced when the targeted alleles are amplified using one or more allele specific primers comprising a nucleotide alteration, for example, a tail.

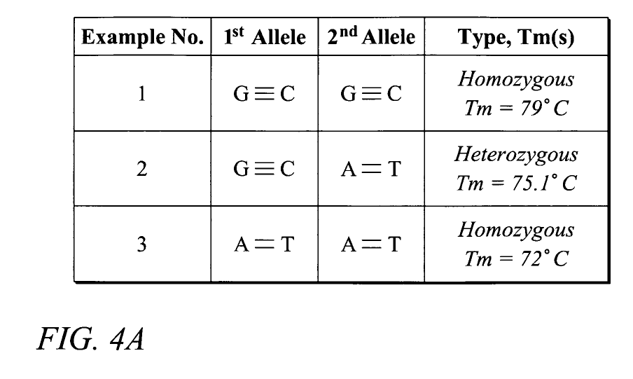

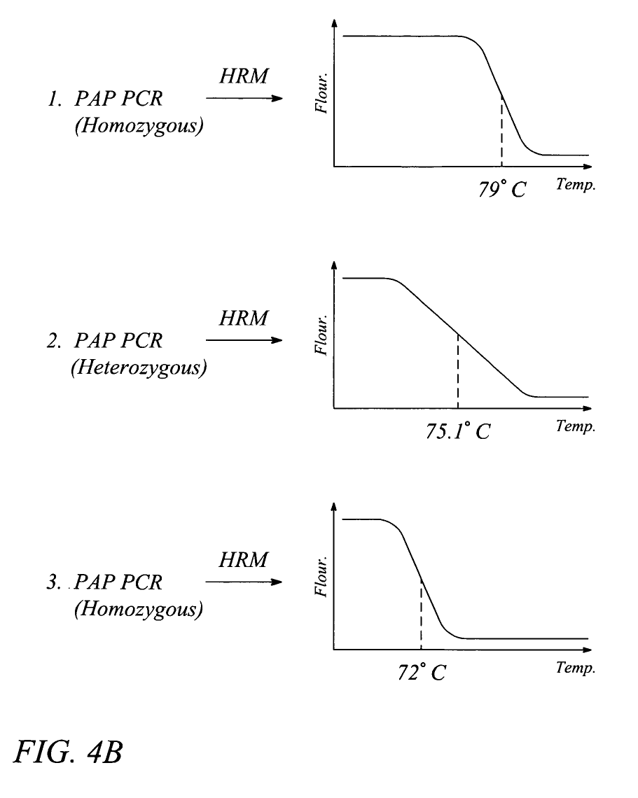

Exemplary melting curves resulting from using tailed allele specific primers to achieve the melting temperatures reported in FIG. 4A are shown in FIG. 4B. The curves resulting from PAP PCR according to various embodiments show Tm curves centered at 72° C., 75.1° C., and 79° C., respectively. The point at which the curve is centered is the mid-point in fluorescence intensity between the initial plateau (or 100 value) on the normalized fluorescence axis (when the amplicons are not melted), and the fluorescence intensity at the curve's lowest level (or 0 value) on the normalized fluorescence axis (when the amplicons are melted).

A Tm of 79° C. is shown in FIG. 4A with regard to Example 1 for amplicons of a sample that is homozygous for the allele comprising the G at the SNP location (a G≡C base pair in the double-stranded form). A Tm of 72° C. is shown in FIG. 4A with regard to Example 3 for amplicons of a sample that is homozygous for the allele comprising the A at the SNP location (an A=T base pair in the double-stranded form). A Tm of 75.1° C. is shown in FIG. 4A with regard to Example 2 for the amplicons of a sample that is heterozygous for both the first and second alleles. As can be seen, there is a significant and easily distinguishable difference between the melting temperature of the SNP amplicons that are homozygous for the double-bonded SNP base pair, versus the melting temperature of the SNP amplicons that are homozygous for the triple bonded SNP base pair, versus the melting temperature of the amplicons of the heterozygous sample. FIG. 4B shows the associated HRM melt curves for each of Examples 1-3 shown in FIG. 4A. When testing an unknown sample to see whether or not the unknown sample is homozygous for the first allele, homozygous for the second allele, or heterozygous, the Tm and curve shape of the HRM melt curve associated with melting amplicons of the unknown sample can be compared to the Tms and curve shapes shown in FIGS. 4A and 4B so that identification and analysis of the unknown sample can be made.

As can be appreciated by one skilled in the art in view of these teachings, the very distinct and separated melt curves facilitate the identification of samples that are homozygous for the first allele, homozygous for the second allele, or heterozygous. According to various embodiments, melting temperature, curve slope, curve shape, or a combination thereof, can be used to distinguish a first homozygous sample from a second homozygous sample, or to distinguish a heterozygous sample from either of two homozygous samples. In some embodiments, initial melting temperature can be used to facilitate genotyping, for example, in some embodiments a heterozygous sample begins to melt before either of the homozygous samples, although the mid-point temperature of the heterozygous sample lies between the mid-point temperatures of the two homozygous samples.

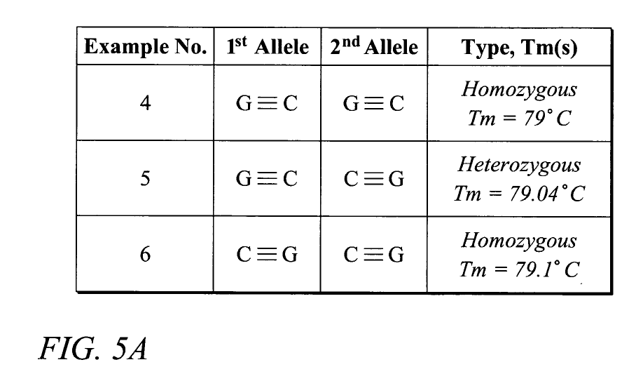

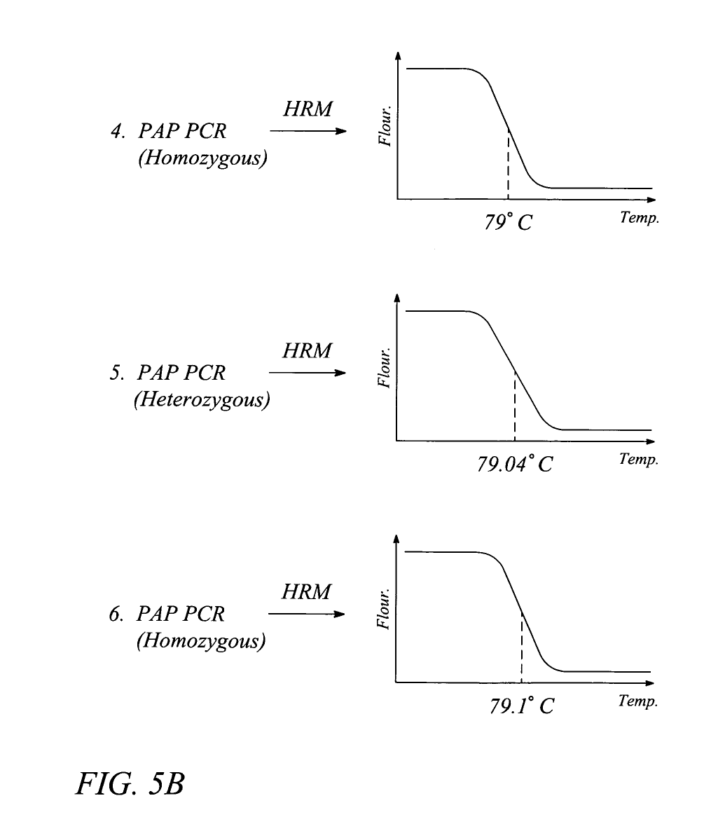

Examples 4-6 shown in FIG. 5A illustrate that when using PAP PCR with allele specific primers that do not contain nucleotide alterations, for example, that do not contain tails, it can be difficult to distinguish between SNP alleles that differ only by a base pair inversion at the SNP location. Distinguishing SNP alleles that differ from one another by only a single base pair inversion can be difficult because the melting temperatures of amplicons of respective homozygous samples of the two alleles differ by only about 0.1° C. As shown in FIG. 5B, it can be difficult to distinguish the melting curves of amplicons of samples that are homozygous for the first allele, from samples that are homozygous for the second allele, and/or from samples that are heterozygous. By using PAP PCR with at least one allele specific primer that comprises a nucleotide alteration, however, the same alleles analyzed in FIG. 5A can be more easily distinguished in view of an enhanced difference in the melting temperatures between amplicons of a sample that is homozygous for the first allele, amplicons of a sample that is homozygous for the second allele, and amplicons of a heterozygous sample.

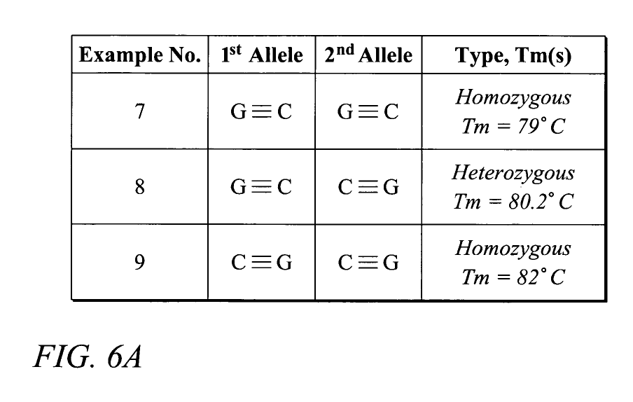

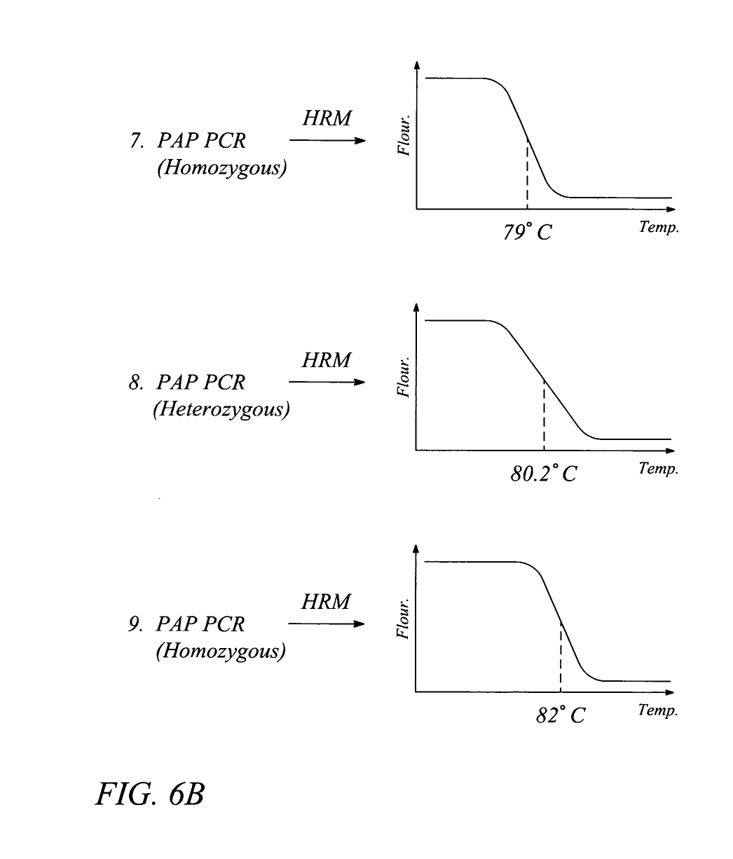

Examples 7-9 shown in FIG. 6A correspond respectively to Examples 4-6 shown in FIG. 5A, but wherein the samples have been hybridized with, and amplified using, at least one allele specific primer that comprises a nucleotide alteration. FIG. 6A illustrates the situation where the allele specific primer that probes for the allele having a C at the SNP location (a C≡G base pair in the double-stranded form) comprises a tail that has caused the melting temperature of the amplicons to increase 2.9° C. such that amplicons from a sample that is homozygous for such an allele have a melting temperature of 82° C. As shown in the melting curves illustrated in FIG. 6B, the difference in melting temperatures (ΔTm) between the two homozygous samples (Examples 7 and 9) is 3° C. as opposed to 0.1° C. for alleles hybridized and amplified using primers without a nucleotide alteration (shown in FIGS. 5A and 5B).

While the present teachings are described in conjunction with various embodiments, it is not intended that the present teachings be limited to such embodiments. On the contrary, the present teachings encompass various alternatives, modifications, and equivalents, as will be appreciated by those of skill in the art.

What is claimed is:

1. A method for analyzing a nucleic acid, comprising:
(a) providing a first allele specific primer and a second allele specific primer, each of the first allele specific primer and the second allele specific primer comprising a single nucleotide polymorphism (SNP) allele-hybridizable sequence, and at least one of the first allele specific primer and the second allele specific primer comprises a nucleotide alteration;
(b) providing a nucleic acid comprising a 3′ end and a SNP base located within 1-20 bases of the 3′ end;
(c) hybridizing at least one of the first allele specific primer and the second allele specific primer to the nucleic acid to form a hybridized nucleic acid;
(d) amplifying the hybridized nucleic acid using pyrophosphorolysis activated polymerization PCR to produce amplicons;
(e) generating a melting curve by melting the amplicons; and
(f) analyzing the nucleic acid based on the melting curve.

2. A method as recited in claim 1, further comprising determining a melting temperature of the amplicons and genotyping the nucleic acid based on the melting temperature.

3. A method as recited in claim 1, further comprising determining a slope of the melting curve and genotyping the nucleic acid based on the slope.

4. A method as recited in claim 3, further comprising determining a melting temperature of the amplicons, wherein the genotyping is also based on the melting temperature.

5. A method as recited in claim 1, further comprising determining a shape of the melting curve and genotyping the nucleic acid based on the shape.

6. A method as recited in claim 1, wherein the amplifying is performed using a pyrophosphorolysis activated polymerization PCR enzyme.

7. The method of claim 1, wherein each of the first allele specific primer and the second allele specific primer comprises a tail comprising one or more nucleotides.

8. A method as recited in claim 1, wherein the nucleotide alteration comprises a tail comprising the sequence GC on at least one of the first allele specific primer and the second allele specific primer.

9. A method as recited in claim 1, wherein the nucleotide alteration comprises a tail comprising the sequence AT on at least one of the first allele specific primer and the second allele specific primer.

10. A method as recited in claim 1, wherein the amplifying comprises using a PCR thermocycler.

11. A method as recited in claim 1, wherein the nucleic acid is double-stranded and comprises up to 60 base pairs.

12. A method as recited in claim 1, wherein the nucleic acid is double-stranded and comprises up to 1000 base pairs.

13. A kit for high resolution melt genotyping, the kit comprising:
(a) a locus specific primer;
(b) one or more allele specific primers each comprising at least one single nucleotide polymorphism (SNP) allele-hybridizable sequence, wherein at least one of the one or more allele specific primers comprises a nucleotide alteration; and
(c) a pyrophosphorolysis activated polymerization polymerase chain reaction (PCR) enzyme.

14. The kit of claim 13, wherein the one or more allelele specific primers comprises two different allele specific primers.

15. A reaction mixture for high resolution melt genotyping, comprising:
(a) a locus specific primer;
(b) one or more allele specific primers each comprising at least one single nucleotide polymorphism (SNP) allele-hybridizable sequence, wherein at least one of the one or more allele specific primers comprises a nucleotide alteration; and
(c) a pyrophosphorolysis activated polymerization polymerase chain reaction (PCR) enzyme.

16. The reaction mixture as recited in claim 15, further comprising a nucleic acid comprising a 3′ end and a single nucleotide polymorphism base located within 1 to 20 bases of the 3′ end.

17. The reaction mixture of claim 15, wherein the one or more allelele specific primers comprises two different allele specific primers.

18. A method comprising:
reacting a locus specific primer, a first allele specific primer, and a second allele specific primer, with a heterozygous nucleic acid sample to form a first hybridized allele and a second hybridized allele, wherein each allele comprises a 3′ end and a single nucleotide polymorphism base located within 1 to 20 bases of the 3′ end; and
amplifying the first hybridized allele and the second hybridized allele using pyrophosphorolysis activated polymerization polymerase chain reaction to form amplicons.

19. The method of claim 18, further comprising performing a melt curve analysis on the amplicons.

20. The method of claim 19, wherein at least one of the first allele specific primer and the second allele specific primer comprises a nucleotide alteration.

21. A method for high resolution melt analysis of a nucleic acid comprising a 3′ end and a single nucleotide polymorphism (SNP) base located within 1-20 bases of the 3′ end, the method comprising:
(a) hybridizing a first allele specific primer to the nucleic acid, wherein the first allele specific primer comprises a sequence adapted to hybridize to a first allele of the nucleic acid, to form a hybridized nucleic acid;
(b) amplifying the hybridized nucleic acid using pyrophosphorolysis activated polymerization (PAP) polymerase chain reaction (PCR) to produce first amplicons; and
(c) determining the melting temperature of the first amplicons by high resolution melt analysis.

22. The method of claim 21, wherein the nucleic acid is heterozygous and comprises at least two different SNP alleleles, the first allele specific primer hybridizes to a first SNP allele of the at least two SNP alleles, and the method further comprises:
(d) hybridizing a second allele specific primer to a second SNP allele of the at least two SNP alleles, to form a second hybridized nucleic acid;
(e) amplifying the second hybridized nucleic acid using PAP PCR to produce second amplicons; and
(f) determining the melting temperature of the combined first and second amplicons by melting curve analysis.

23. The method of claim 22, further comprising correlating the melting temperature analysis of the combined first and second amplicons to the heterozygosity of the nucleic acid.

24. The method of claim 22, further comprising hybridizing a locus specific primer to the nucleic acid before amplifying the hybridized nucleic acid.

25. The method of claim 22, wherein the melting curve analysis comprises a high resolution melt analysis.
